(19) United States
(12) Patent Application Publication
(10) Pub. No.: US 2022/0090065 A1
(43) Pub. Date: Mar. 24, 2022

(54) Novel tracrRNA system for Cas9

(71) Applicant: KOREA INSTITUTE OF SCIENCE AND TECHNOLOGY, Seoul (KR)

(72) Inventors: Dae-Ro AHN, Seoul (KR); Jihyun PARK, Seoul (KR); Hak Suk CHUNG, Seoul (KR); Cherlhyun JEONG, Seoul (KR)

(21) Appl. No.: 17/464,691

(22) Filed: Sep. 2, 2021

(30) Foreign Application Priority Data
Sep. 2, 2020 (KR) 10-2020-0111923

(51) Int. Cl. C12N 15/11; C12N 9/22

(52) U.S. Cl. CPC C12N 15/111; C12N 2310/20; C12N 9/22

(57) ABSTRACT

The present invention relates to a novel trans-activating CRISPR RNA (tracrRNA) including a fragment of a tracrRNA, and a gRNA, a CRISPR-Cas9 system, and a kit including the tracrRNA.

### TECHNICAL FIELD

The present invention relates to a novel tracrRNA system including a fragment of a tracrRNA, and a gRNA and a CRISPR-Cas9 system including the novel tracrRNA system.

### BACKGROUND ART

Gene editing is a biotechnology that enables knock-in, a gene technology that knocks out a target gene function by cleaving a specific gene site or substitutes a knocked-out gene with a desired gene. The CRISPR-Cas9 system, developed through the first generation ZFN and second generation TALEN, is a third-generation genetic scissors system, and is widely used because of its superior specificity and the fact that it is not necessary to create a new DNA binding region every time (Zhang, F. et al., CRISPR/Cas9 for genome editing: progress, implications and challenges. Human Molecular Genetics, 2014, 23(R1), R40-R46)).

Such CRISPR-Cas9 system is different from the first and second generations of gene scissors in that gene cleavage is performed as a specific nucleotide sequence consisting of 3 to 4 bases around the target gene is recognized by a guide RNA (gRNA). As a specific gRNA, single gRNA (sgRNA) or double gRNA has been used, but problems such as the practicality of the two types of gRNAs have continuously arisen.

For example, existing sgRNA has been used as a ribonucleoprotein (RNP) complex in which the sgRNA is combined with a Cas9. The sgRNA and Cas9 have each been produced by being cloned with a DNA vector and expressed. However, in this case, problems have arisen in that the use of a plasmid vector decreases the Cas9 activity and causes an off-target effect and intracellular toxicity. Since the sgRNA consists of a long sequence having a total length of more than 100 nucleotides, the synthesis yield is significantly low, and mass-production is difficult.

In addition, the dual-crRNA:tracrRNA gRNA system, in which two gRNAs produced by way of chemical synthesis are used, also has disadvantages in that the tracrRNA generally consists of a long RNA sequence of 89 nucleotides, and this significantly hinders mass-production, but the development of efficient gRNAs is not sufficiently carried out.

With this background, the present inventors have made diligent efforts to develop gRNAs that are more convenient than the existing single and double gRNA systems, can be mass-produced, can thus be widely applied practically, and can maintain the sensitivity to the target sequence, and as a result, they have developed a CRISPR-Cas9 system utilizing a fragment of a general tracrRNA or a combination thereof as a tracrRNA, thereby completing the present invention.

### DISCLOSURE

### Technical Problem

An object of the present invention is to provide a trans-activating CRISPR RNA (tracrRNA).

Another object of the present invention is to provide a guide RNA (gRNA) including the tracrRNA.

Still another object of the present invention is to provide a CRISPR-Cas9 system including the tracrRNA.

Still another object of the present invention is to provide a kit for DNA expression inhibition and DNA targeting.

Still another object of the present invention is to provide a kit for gene editing.

### Technical Solution

Hereinafter, the contents of the present invention will be described in detail as follows. Meanwhile, descriptions and embodiments of one aspect disclosed in the present invention may also be applied to descriptions and embodiments of other aspects with regard to common matters. Moreover, all combinations of the various elements disclosed in the present invention are within the scope of the present invention. In addition, it cannot be seen that the scope of the present invention is limited by the specific descriptions described below.

An aspect of the present invention for achieving the objects provides a trans-activating CRISPR RNA (tracrRNA) including a nucleotide sequence of SEQ ID NO: 2.

As used herein, the term “tracrRNA” (trans-activating CRISPR RNA) refers to an RNA that binds to a crRNA to form a guide RNA (gRNA), and is a component of a CRISPR-Cas9 system.

A wild-type tracrRNA is generally a polynucleotide having a size of 75 nucleotides or more. However, in the present invention, it has been confirmed that a tracrRNA (67), which is a part of the wild-type tracrRNA and consists of the nucleotide sequence (67 nucleotides) of SEQ ID NO: 1, also performs the function of a tracrRNA (Examples 3 and 4), and this is a result corresponding to the previously known function of a tracrRNA. However, for the purpose of the present invention, the tracrRNA functions as a tracrRNA while including a nucleotide sequence of SEQ ID NO: 2, and may have a shorter sequence than the wild-type tracrRNA or tracrRNA (67). The tracrRNA may further include one or more fragments of the nucleotide sequence of SEQ ID NO: 1, specifically one or two fragments of the nucleotide sequence of SEQ ID NO: 1, and more specifically one fragment of the nucleotide sequence of SEQ ID NO: 1. The tracrRNA further including one nucleotide sequences of SEQ ID NO: 1 is a tracrRNA including two fragments of the nucleotide sequence of SEQ ID NO: 1, and one or more of the two fragments may include the nucleotide sequence of SEQ ID NO: 2.

As used herein, the term “fragment” refers to a partial sequence of a polynucleotide having a specific sequence, and specifically a partial sequence of the wild-type tracrRNA or tracrRNA (67) and a sequence of a partial region that may be utilized by being included in the tracrRNA. The sequence or length is not limited as long as it is a partial sequence of the wild-type tracrRNA or tracrRNA (67) and is included in the tracrRNA to exhibit activity in a Cas9 system. It is apparent that sequences in which meaningless sequences are added to both ends of the fragment or in which a part of the sequence is substituted are also included in the scope of the present invention.

Specifically, the fragment may be a fragment of the wild-type tracrRNA or a fragment of SEQ ID NO: 1 (tracrRNA (67)), and specifically may include the nucleotide sequence of any one of SEQ ID NOs: 2 to 27. More specifically, the fragment including the nucleotide sequence of SEQ ID NO: 2 may be any one of SEQ ID NOs: 2, 4, 6, 8, 13, 15, 17, 19, 21, 23, 25, and 27, but is not limited thereto.

The size of the fragment may be 15 nucleotides or more and 50 nucleotides or less, specifically, 18 nucleotides or more and 49 nucleotides or less, 18 nucleotides or more and 44 nucleotides or less, 18 nucleotides or more and 43 nucleotides or less, 18 nucleotides or more and 42 nucleotides or less, 18 nucleotides or more and 41 nucleotides or less, 18 nucleotides or more and 40 nucleotides or less, 18 nucleotides or more and 39 nucleotides or less, 18 nucleotides or more and 38 nucleotides or less, 18 nucleotides or more and 37 nucleotides or less, 18 nucleotides or more and 36 nucleotides or less, 18 nucleotides or more and 35 nucleotides or less, 18 nucleotides or more and 34 nucleotides or less, 18 nucleotides or more and 33 nucleotides or less, 18 nucleotides or more and 32 nucleotides or less, 18 nucleotides or more and 31 nucleotides or less, 18 nucleotides or more and 30 nucleotides or less, 18 nucleotides or more and 29 nucleotides or less, 18 nucleotides or more and 28 nucleotides or less, 18 nucleotides or more and 27 nucleotides or less, 18 nucleotides or more and 26 nucleotides or less, 18 nucleotides or more and 25 nucleotides or less, 18 nucleotides or more and 24 nucleotides or less, 18 nucleotides or more and 23 nucleotides or less, 26 nucleotides or more and 44 nucleotides or less, 26 nucleotides or more and 43 nucleotides or less, 26 nucleotides or more and 42 nucleotides or less, or 26 nucleotides or more and 41 nucleotides or less, but is not limited thereto. The main sequence of the fragment may be 18 polynucleotides from the 5′ end; 18 polynucleotides from the 3′ end; or 20 polynucleotides from the 3′ end of the tracrRNA consisting of the nucleotide sequence of SEQ ID NO: 1, but is not limited thereto.

Specifically, in the present invention, it has been confirmed that a tracrRNA including a fragment containing a region where the wild-type tracrRNA or tracrRNA (67) binds to a crRNA can function as a tracrRNA similarly to the wild-type tracrRNA. It has been confirmed that the region complementarily binding to a crRNA is a region consisting of the nucleotide sequence of SEQ ID NO: 2 (region 1 in FIG. 1), and contains the region consisting of the nucleotide sequence of SEQ ID NO: 2 as long as it can function as a tracrRNA as a tracrRNA including a tracrRNA having a shorter length than a wild-type tracrRNA, a fragment of the wild-type tracrRNA, and a fragment of the tracrRNA. It has been confirmed that the region consisting of the nucleotide sequence of SEQ ID NO: 2 is an essential region to act as a tracrRNA.

In an embodiment, the tracrRNA may include a fragment consisting of the nucleotide sequence of SEQ ID NO: 4 (A in FIG. 1), a fragment consisting of the nucleotide sequence of SEQ ID NO: 6; a fragment consisting of the nucleotide sequence of SEQ ID NO: 8; a fragment consisting of the nucleotide sequence of SEQ ID NO: 4 and a fragment consisting of the nucleotide sequence of SEQ ID NO: 11 (A+3 in FIG. 1); a fragment consisting of the nucleotide sequence of SEQ ID NO: 4 and a fragment consisting of the nucleotide sequence of SEQ ID NO: 9 (A+4 in FIG. 1); a fragment consisting of the nucleotide sequence of SEQ ID NO: 4, a fragment consisting of the nucleotide sequence of SEQ ID NO: 11, and a fragment consisting of the nucleotide sequence of SEQ ID NO: 9 (A+3+4 in FIG. 1); a fragment consisting of the nucleotide sequence of SEQ ID NO: 2 and a fragment consisting of the nucleotide sequence of SEQ ID NO: 3 (FIG. 2); a fragment consisting of the nucleotide sequence of SEQ ID NO: 4 and a fragment consisting of the nucleotide sequence of SEQ ID NO: 5 (A+B in FIG. 1); a fragment consisting of the nucleotide sequence of SEQ ID NO: 6 and a fragment consisting of the nucleotide sequence of SEQ ID NO: 7 (FIG. 2); and/or a fragment consisting of the nucleotide sequence of SEQ ID NO. 8 and a fragment consisting of the nucleotide sequence of SEQ ID NO: 9 (FIG. 2), but is not limited thereto.

In an embodiment, the tracrRNA may include a fragment consisting of the nucleotide sequence of SEQ ID NO: 13, a fragment consisting of the nucleotide sequence of SEQ ID NO: 15; a fragment consisting of the nucleotide sequence of SEQ ID NO: 17; a fragment consisting of the nucleotide sequence of SEQ ID NO: 19; a fragment consisting of the nucleotide sequence of SEQ ID NO: 21; a fragment consisting of the nucleotide sequence of SEQ ID NO: 23; a fragment consisting of the nucleotide sequence of SEQ ID NO: 25; a fragment consisting of the nucleotide sequence of SEQ ID NO: 27; a fragment consisting of the nucleotide sequence of SEQ ID NO: 13 and a fragment consisting of the nucleotide sequence of SEQ ID NO: 14; a fragment consisting of the nucleotide sequence of SEQ ID NO: 15 and a fragment consisting of the nucleotide sequence of SEQ ID NO: 16; a fragment consisting of the nucleotide sequence of SEQ ID NO: 17 and a fragment consisting of the nucleotide sequence of SEQ ID NO: 18; and/or a fragment consisting of the nucleotide sequence of SEQ ID NO: 19 and a fragment consisting of the nucleotide sequence of SEQ ID NO. 20, but is not limited thereto.

In an embodiment, the tracrRNA may include a fragment consisting of the nucleotide sequence of SEQ ID NO: 13 and a fragment consisting of the nucleotide sequence of SEQ ID NO: 7, a fragment consisting of the nucleotide sequence of SEQ ID NO: 13 and a fragment consisting of the nucleotide sequence of SEQ ID NO: 16; a fragment consisting of the nucleotide sequence of SEQ ID NO: 13 and a fragment consisting of the nucleotide sequence of SEQ ID NO: 18; a fragment consisting of the nucleotide sequence of SEQ ID NO: 13 and a fragment consisting of the nucleotide sequence of SEQ ID NO: 20; a fragment consisting of the nucleotide sequence of SEQ ID NO: 6 and a fragment consisting of the nucleotide sequence of SEQ ID NO: 16; a fragment consisting of the nucleotide sequence of SEQ ID NO: 6 and a fragment consisting of the nucleotide sequence of SEQ ID NO: 18; a fragment consisting of the nucleotide sequence of SEQ ID NO: 6 and a fragment consisting of the nucleotide sequence of SEQ ID NO: 20; a fragment consisting of the nucleotide sequence of SEQ ID NO: 15 and a fragment consisting of the nucleotide sequence of SEQ ID NO: 18; a fragment consisting of the nucleotide sequence of SEQ ID NO: 15 and a fragment consisting of the nucleotide sequence of SEQ ID NO: 20; and/or a fragment consisting of the nucleotide sequence of SEQ ID NO: 17 and a fragment consisting of the nucleotide sequence of SEQ ID NO: 20, but is not limited thereto.

In an embodiment, the tracrRNA may include a fragment consisting of the nucleotide sequence of SEQ ID NO: 21 and a fragment consisting of the nucleotide sequence of SEQ ID NO: 22, a fragment consisting of the nucleotide sequence of SEQ ID NO: 23 and a fragment consisting of the nucleotide sequence of SEQ ID NO: 24; a fragment consisting of the nucleotide sequence of SEQ ID NO: 25 and a fragment consisting of the nucleotide sequence of SEQ ID NO: 26; and/or a fragment consisting of the nucleotide sequence of SEQ ID NO: 27 and a fragment consisting of the nucleotide sequence of SEQ ID NO: 26, but is not limited thereto.

In the present invention, a tracrRNA including two or more fragments may be in a form in which the respective fragments are linked to each other, or the respective fragments are not linked to each other but are adjacent to each other in a three-dimensional structure and adjacent to each other in a Cas9 system so as to exhibit a characteristic of recognizing a target sequence, but is not limited thereto.

Specifically, it has been confirmed that a tracrRNA including a fragment containing the nucleotide sequence of SEQ ID NO: 2 has a small size, convenience, practicality, and economical efficiency, and at the same time, allows a CRISPR-Cas9 system to exhibit as high sensitivity as a wild-type tracrRNA introduced CRISPR-Cas9 system when the tracrRNA is introduced into the CRISPR-Cas9 system (Examples 3 and 4).

As used herein, the term “CRISPR-Cas9” system refers to a third generation genetic scissors, and refers to a nucleotide sequence-specific endonuclease system formed by the binding of a gRNA and a Cas9 protein.

As used herein, the term “gRNA” (guide RNA) refers to an RNA that serves to induce the specificity of a DNA sequence of a target region and cleave a target gene specifically to a nucleotide sequence. The gRNA may be divided into sgRNA (single guide RNA) and dual gRNA (dual guide RNA) systems according to the number of RNAs constituting the gRNA, and a dual gRNA is generally formed by the binding of a crRNA (CRISPR RNA) and a tracrRNA and may be thus used interchangeably with crRNA: tracrRNA or dual-crRNA: tracrRNA.

As used herein, the term “Cas9” (CRISPR-associated protein9) refers to a protein binding to a gRNA to form a CRISPR-Cas9 system.

In the present invention, even if a DNA, RNA, polynucleotide, gene or protein is described as having the nucleotide sequence or amino acid sequence of a specific SEQ ID NO, it is apparent that a DNA, RNA, polynucleotide, gene or protein in which a part of the sequence is deleted, modified, substituted, conservatively substituted or added may also be used in the present invention as long as it exhibits the same or corresponding activity as the DNA, RNA, polynucleotide, gene or protein consisting of the nucleotide sequence or amino acid sequence of the corresponding SEQ ID NO.

As long as it exhibits the same or corresponding activity as a DNA, RNA, polynucleotide, gene or protein of the nucleotide sequence or amino acid sequence represented by a specific SEQ ID NO, a sequence having 80% or more homology or identity with the nucleotide sequence or amino acid sequence represented by the specific SEQ ID NO may also be included in the scope of the present invention, but the sequence is not limited thereto. Specifically, it is apparent that a sequence, which may have at least 80%, 85%, 90%, 95%, 96%, 97%, 98%, or 99% or more homology or identity with the nucleotide sequence or amino acid sequence of a specific SEQ ID NO and exhibits the corresponding efficacy or activity and of which a part is deleted, modified, substituted, or added, is also included in the scope of the present invention.

As used herein, the term ‘homology’ or ‘identity’ refers to the degree to which two given amino acid sequences or nucleotide sequences are related to each other and may be expressed as a percentage.

The terms homology and identity may often be used interchangeably.

The sequence homology or identity of a conserved polynucleotide or polypeptide is determined using a standard alignment algorithm, and a default gap penalty established by the program being used may be used together. Substantially homologous or identical sequences are generally capable of being hybridized at least at about 50%, 60%, 70%, 80%, or 90% or more of the entire or full-length sequence under moderate or high stringent conditions. Hybridization is also contemplated for polynucleotides containing degenerate codons instead of codons in the polynucleotides.

Whether arbitrary two polynucleotide or polypeptide sequences have homology, similarity, or identity may be determined, for example, using a default parameter as in Pearson et al. (1988) Proc. Natl. Acad. Sci. USA 85:2444 and a known computer algorithm, such as the “FASTA” program. Alternatively, the Needleman-Wunsch algorithm (Needleman and Wunsch, 1970, J. Mol. Biol. 48:443-453) as performed in the Needleman program of the EMBOSS package (EMBOSS: The European Molecular Biology Open Software Suite, Rice et al, 2000, Trends Genet. 16:276-277) (version 5.0.0 or later) may be used to determine the homology, similarity, or identity. (The GCG program package (Devereux, J., et al., Nucleic Acids Research 12:387 (1984)), BLASTP, BLASTN, FASTA (Atschul, S. F., et al, J MOLEC BIOL 215:403 (1990); Guide to Huge Computers, Martin J. Bishop, ed., Academic Press, San Diego, 1994, and CARILLO et al. (1988) SIAM J Applied Math 48:1073) are included). For example, BLAST from the National Center for Biotechnology Information Database, or ClustalW may be used to determine the homology, similarity, or identity.

The homology, similarity, or identity of polynucleotides or polypeptides may be determined by comparing the sequence information, for example, using a GAP computer program such as Needleman et al., (1970) J Mol Biol. 48:443, for example, as known in Smith and Waterman, Adv. Appl. Math (1981) 2:482. In summary, in the GAP program, the homology, similarity, or identity is defined as the value acquired by dividing the number of similarly aligned symbols (namely, nucleotides or amino acids) by the total number of symbols in the shorter of two sequences. The default parameters for GAP programs may include (1) unary comparison matrix (containing values of 1 for identity and 0 for non-identity) and weighted comparison matrix of Gribskov et al. (1986) Nucl. Acids Res. 14:6745 (or EDNAFULL (EMBOSS version of NCBI NUC4.4) substitution matrix) as disclosed in Schwartz and Dayhoff, eds., Atlas Of Protein Sequence And Structure, National Biomedical Research Foundation, pp. 353-358 (1979); (2) a penalty of 3.0 for each gap and an additional penalty of 0.10 for each symbol in each gap (or a gap opening penalty of 10, and a gap extension penalty of 0.5); and (3) no penalty for an end gap. Hence, as used herein, the term “homology” or “identity” refers to the relatedness between sequences.

As used herein, the term “complementary” is used to describe the relation between nucleotide bases capable of being hybridized with each other. For example, with regard to DNA, adenosine is complementary to thymine and cytosine is complementary to guanine. Hence, the present invention may also include substantially similar nucleic acid sequences as well as isolated nucleic acid fragments complementary to the entire sequence.

Another aspect of the present invention provides a guide RNA (gRNA) including the tracrRNA. The gRNA may further include a CRISPR RNA (crRNA), and the crRNA may be an RNA consisting of the nucleotide sequence of SEQ ID NO: 12 for the purposes of the present invention, but is not limited thereto. The terms fragment, tracrRNA, crRNA, and gRNA are as described above. Specifically, the tracrRNA, crRNA, and/or gRNA may include one or more modified nucleotides, and more specifically, the modified nucleotide may be any one selected from the group consisting of 2′-O-methyl RNA (2′-OMe RNA), 2′-O-methoxyethyl RNA (2′-MOE RNA), 2′-fluoro RNA (2′-F RNA), phosphorothioate RNA (PS RNA), 2′-amino RNA (2′-NH₂ RNA), 2′-fluoro arabinose nucleic acid (FANA), 4′-thio RNA (4′-S RNA), locked nucleic acid (LNA), threose nucleic acid (TNA), phosphorothioate DNA (PS DNA), DNA, peptide nucleic acid (PNA), phosphorodiamidate morpholino oligonucleotide (PMO), hexitol nucleic acid (HNA), cyclohexene nucleic acid (CeNA), and arabinose nucleic acid (ANA).

The tracrRNA of the present invention may have a stabilized structure. For example, the stabilization may be to stabilize the double chain binding force by changing the sugar backbone, to contain a stem-loop, or to contain guanine (G) or cytosine (C), and the guanine or cytosine may be contained before and after the stem-loop, but the stabilization is not limited thereto. The change in the sugar backbone may be to use 2′-fluoro-RNA, 2′-O-methyl-RNA (2′-OMe-RNA), LNA (locked nucleic acid), PNA (peptide nucleic acid) or the like instead of an RNA, but is not limited thereto.

Still another aspect of the present invention provides a CRISPR-Cas9 system including the tracrRNA. The terms fragment, tracrRNA, and CRISPR-Cas9 system are as described above.

Still another aspect of the present invention provides a kit for DNA expression inhibition and DNA targeting, the kit including the tracrRNA described above or a DNA encoding the tracrRNA; a crRNA or a DNA encoding the crRNA; and a Cas9 protein or a polynucleotide encoding the Cas9 protein. The terms fragment, tracrRNA, crRNA, and Cas9 are as described above.

The operating principle or form of the kit is not limited as long as the kit can inhibit DNA expression and target DNA. The kit of the present invention may further include one or more other components, compositions, solutions or devices suitable for DNA expression inhibition and DNA targeting, but is not limited thereto. Examples of the other components include, but are not limited to, suitable carriers, solubilizers, buffers, and stabilizers. The carriers include soluble carriers and insoluble carriers, examples of the soluble carriers include a physiologically acceptable buffer known in the art, for example, PBS, and examples of the insoluble carriers include polymers such as polystyrene, polyethylene, polypropylene, polyester, polyacrylonitrile, fluororesin, cross-linked dextran, polysaccharide, and magnetic fine particles in which latex is plated with a metal, other paper, glass, metals, agarose, and combinations thereof, but the carrier is not limited thereto.

The kit of the present invention may further include a user guide describing optimal conditions for conducting the reaction. The guide is a printed document explaining the method of using the kit, for example, the method of preparing the buffer and the reaction conditions presented. The guide includes a brochure in the form of a pamphlet or leaflet, a label affixed to the kit, and instructions on the surface of the package containing the kit. The guide contains information that is published or provided through electronic media such as the Internet.

Still another aspect of the present invention provides a kit for gene editing, the kit including the tracrRNA described above or a DNA encoding the tracrRNA; a crRNA or a DNA encoding the crRNA; and a Cas9 polypeptide or a polynucleotide encoding the Cas9 polypeptide. The terms fragment, tracrRNA, crRNA, Cas9, and kit are as described above.

### Advantageous Effects

A tracrRNA system using the short tracrRNA of the present invention as well as a tracrRNA system including a fragment thereof can be applied as a tracrRNA system equipped with all of the simplicity, economical efficiency and practicality while maintaining the sensitivity of the existing system. From this, the tracrRNA can be expected to be industrially utilized in the function identification of genetic information, gene editing, and disease treatment.

### BRIEF DESCRIPTION OF DRAWINGS

FIG. 1 is a diagram illustrating the confirmed essential region of a fragment of SEQ ID NO: 1;

FIGS. 2A and 2B are diagrams illustrating a schematic diagram of the sequence of a tracrRNA including SEQ ID NO: 1 or a fragment thereof;

FIG. 3 is a diagram illustrating the in vitro target cleavage confirmation results of a CRISPR-Cas9 system including SEQ ID NO: 1 or a fragment thereof in a tracrRNA;

FIGS. 4 to 6 are diagrams illustrating the off-target evaluation results of a CRISPR-Cas9 system including SEQ ID NO: 1 or a fragment thereof in a tracrRNA;

FIG. 7 is a diagram illustrating the target cleavage activity confirmation (flow cytometry) results of a CRISPR-Cas9 system including SEQ ID NO: 1 or a fragment thereof in a tracrRNA;

FIG. 8 is a diagram illustrating the target cleavage activity confirmation (Western blot) results of a CRISPR-Cas9 system including SEQ ID NO: 1 or a fragment thereof in a tracrRNA;

FIG. 9 is a diagram illustrating the target cleavage activity confirmation (T7E1 assay) results of a CRISPR-Cas9 system including SEQ ID NO: 1 or a fragment thereof in a tracrRNA;

FIG. 10 is a diagram illustrating the target cleavage activity confirmation (TIDE analysis) results of a CRISPR-Cas9 system including SEQ ID NO: 1 or a fragment thereof in a tracrRNA;

FIG. 11 is a diagram illustrating that the synthesis yield of an oligonucleotide decreases as the length of the oligonucleotide increases;

FIG. 12 is a diagram illustrating the in vitro target cleavage assay results of a CRISPR-Cas9 system including SEQ ID NO: 1 or a fragment thereof in a tracrRNA;

FIGS. 13A and 13B are diagrams illustrating the off-target evaluation results of a CRISPR-Cas9 system including SEQ ID NO: 1 or a fragment thereof in a tracrRNA;

FIG. 14 is a diagram illustrating the target cleavage activity confirmation (flow cytometry) results of a CRISPR-Cas9 system including SEQ ID NO: 1 or a fragment thereof in a tracrRNA;

FIG. 15 is a diagram illustrating the target cleavage activity confirmation (TIDE analysis) results of a CRISPR-Cas9 system including SEQ ID NO: 1 or a fragment thereof in a tracrRNA;

FIG. 16 is a diagram illustrating a schematic diagram of the sequence of a tracrRNA including a fragment of SEQ ID NO: 1;

FIG. 17 is a diagram illustrating the in vitro target cleavage assay results of a CRISPR-Cas9 system including SEQ ID NO: 1 or a fragment thereof in a tracrRNA;

FIG. 18 is a diagram illustrating the target cleavage activity confirmation (flow cytometry) results of a CRISPR-Cas9 system including SEQ ID NO. 1 or a fragment thereof in a tracrRNA;

FIG. 19 is a diagram illustrating the target cleavage activity confirmation (TIDE analysis) results of a CRISPR-Cas9 system including SEQ ID NO: 1 or a fragment thereof in a tracrRNA;

FIG. 20 is a diagram illustrating the in vitro target cleavage assay results of a CRISPR-Cas9 system using a tracrRNA in which SEQ ID NO: 1 or a fragment thereof is included and a part of the sequence of stem-loop 2 is deleted;

FIG. 21 is a diagram illustrating a schematic diagram of the sequence of a tracrRNA including a sequence in which stem-loop 2 is removed from a fragment combination of SEQ ID NO: 1 and then some of its adjacent nucleotides are substituted with cytosine or guanine;

FIG. 22 is a diagram illustrating the in vitro target cleavage assay results of a CRISPR-Cas9 system using a tracrRNA including a sequence in which stem-loop 2 is removed from a fragment combination of SEQ ID NO: 1 and then some of its adjacent nucleotides are substituted with cytosine or guanine;

FIG. 23 is a diagram illustrating the target cleavage activity confirmation (flow cytometry) results of a CRISPR-Cas9 system using a tracrRNA including a sequence in which stem-loop 2 is removed from a fragment combination of SEQ ID NO: 1 and then some of its adjacent nucleotides are substituted with cytosine or guanine; and

FIG. 24 is a diagram illustrating the target cleavage activity confirmation (TIDE analysis) results of a CRISPR-Cas9 system using a tracrRNA including a sequence in which stem-loop 2 is removed from a fragment combination of SEQ ID NO: 1 and then some of its adjacent nucleotides are substituted with cytosine or guanine.

### DETAILED DESCRIPTION OF THE INVENTION

Hereinafter, the present invention will be described in detail with reference to Examples, but this is only to help the understanding of the present invention, and the scope of the present invention is not limited thereto.

### Example 1: Obtaining of DNA, crRNA, Wild-Type tracrRNA, and Cas9

### Example 1-1: Obtaining of DNA, crRNA, and Wild-Type tracrRNA

DNA, CRISPR RNA (crRNA), and chimeric oligonucleotides were purchased from Integrated DNA Technologies (USA) and Bioneer (Korea). As wild-type transactivating crRNA (tracrRNA), Alt-R® CRISPR-Cas9 tracrRNA (Integrated DNA Technologies, USA) was used.

### Example 1-2: Preparation of Cas9

pET-NLS-Cas9-6×His was purchased from Addgene (USA, plasmid #62934) and purified. Thereafter, pellets were incubated, resuspended in buffer A (50 mM Tris-HCl, pH 8.0, 0.5 M NaCl, 20 mM imidazole, and 5 mM 2-mercaptoethanol (Bio-rad)) and lysed by sonication. After centrifugation at 10,000 g for 40 minutes at 4° C., NLS-Cas9 was separated using Ni-NTA affinity chromatography. The eluted Cas9 was loaded onto Hiprep SP HP 16/10 column (GE Health-care Life Sciences) and purified using a linear gradient of KCl from 0.15 M to 1 M in buffer B (20 mM HEPES, pH 7.5, 10% glycerol, and 2 mM DTT). The eluted protein was concentrated, flash-frozen in liquid nitrogen, and stored at −80° C. The final purity and concentration of Cas9 was determined via a SDS PAGE gel and Bradford protein assay (Bio-Rad, USA) using BSA (bovine serum albumin) as a protein standard, respectively. Catalytically inactive mutant dCas9 (D10A/H840A) was generated by Quick change site-directed mutagenesis according to the manufacturer's protocol (Agilent, USA).

### Example 2: tracrRNA Function Evaluation Method

In order to evaluate the function of the discovered tracrRNA, the present inventors prepared an RNP complex including the tracrRNA, namely, a CRISPR-Cas9. With regard to the prepared RNP complex, in vitro target cleavage activity was confirmed and extracellular off-target effect analysis was performed, and at the intracellular level, intracellular transfection (on/off target) and target cleavage activity confirmation (target gene knockout confirmation; flow cytometry, Western blot, T7E1 assay, and TIDE analysis) were performed. Flow cytometry and Western blot were performed by way of general known methods, and the other methods were performed as follows.

### Example 2-1: Confirmation of In Vitro Target Cleavage Activity (In Vitro Target Cleavage Assay, In Vitro Cas9 Activity Assay)

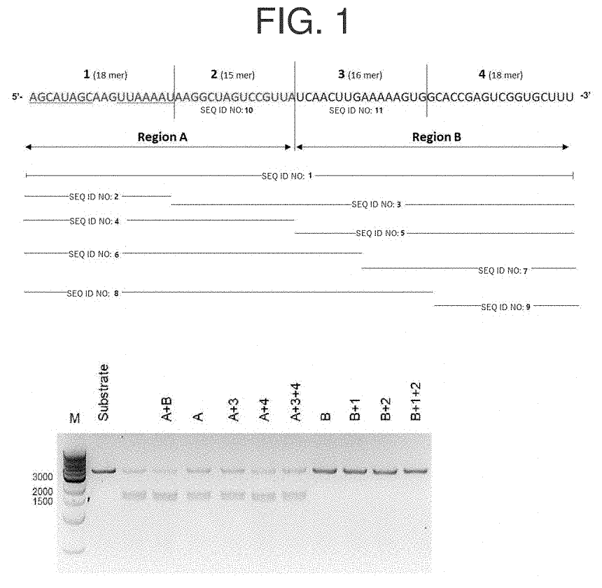

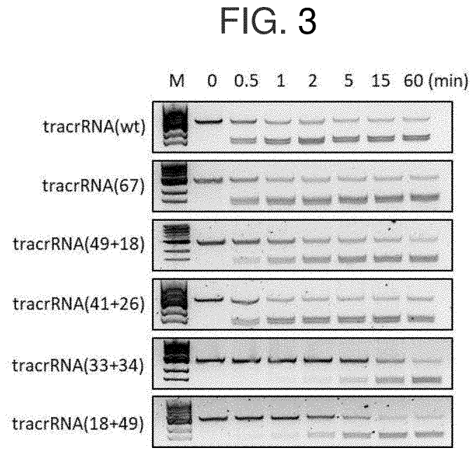

The pSMART-EGFP plasmid encoding a green fluorescent protein (GFP) gene was kindly provided by the University of Seoul and used as a DNA substrate for in vitro DNA cleavage assays. The reaction mixtures (30 μL) containing Cas9 (33 nM), linearized pSMART-EGFP substrate (1 nM) crRNA (33 nM), and tracrRNA (33 nM) in the reaction buffer (20 mM HEPES pH 6.5, 100 mM NaCl, 5 mM MgCl₂, 0.1 mM EDTA) were incubated at 37° C. for 10 or 60 minutes. Thereto, 6× gel loading buffer (19.8 mM Tris-HCl, pH 8.0, 66 mM EDTA, 0.017% SDS, 2.5% Ficoll®-400, 0.015% bromophenol blue, 5 μL, New England Biolabs, USA) was added, and analysis was then performed by way of agarose gel (0.7%) electrophoresis. Gels were stained with SYBR gold (Life Technologies, USA) and imaged with ChemiDoc XRS+system (Bio-rad). Band intensities were quantified using ImageJ (FIGS. 1 and 3 and the like). The cleavage level was determined in the following manner, and the data represent the averaged values and standard deviation of three independent experiments.

Cleavage level (%)=100×[sum of band intensities of cleavage products]/[sum of all band intensities]

### Example 2-2: Off Target Effect Analysis

The off-target effect refers to a side effect in which gene mutations are induced at locations completely different from the expected locations as the gene scissors operate at the location with the desired DNA sequence in some cases.

In order to analyze the off-target effect, a plasmid structure inducing a point mutation in the EGFP gene was prepared, single mutations, in which only one nucleotide sequence was changed, and double and triple mutations, in which two or more nucleotide sequences were changed, were prepared together, and the target sequence sensitivities of the respective RNP complexes were compared with each other.

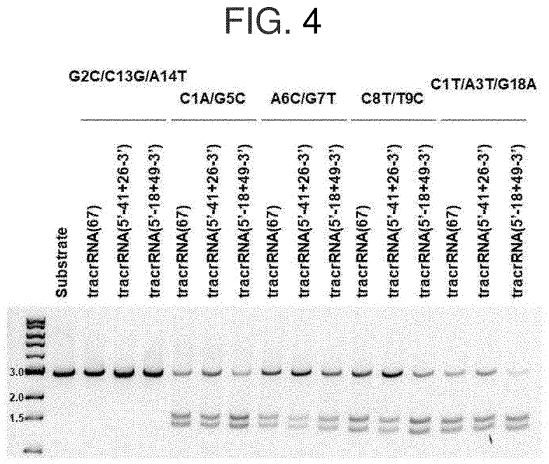

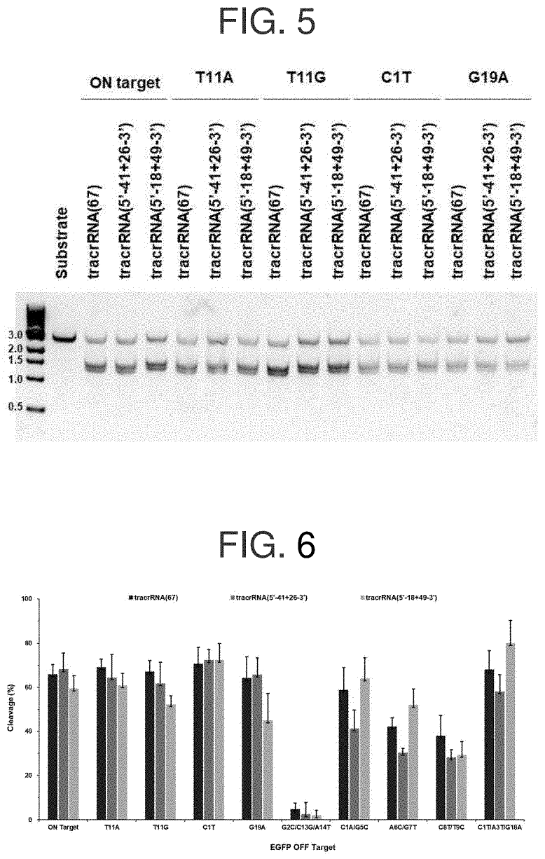

The mutant pSMART-EGFP plasmids containing off-targets were prepared by mutagenesis of a pSMART-EGFP plasmid substrate using Q5 site-directed mutagenesis kit (New England Biolabs, USA). The sequences of mutant plasmids were confirmed via DNA sequencing (Macrogene, Korea). The off-target effect was evaluated by treating such mutant plasmids with a CRISPR/Cas9 system (FIGS. 4 to 6 and the like).

### Example 2-3: Confirmation of Target Cleavage Activity (Flow Cytometry)

In order to confirm the target cleavage activity at the cellular level, the Hela-EGFP/RFP cell line was utilized. Hela/EGFP/RFP cells (1×10⁵) were seeded in 12-well plates and cultured in DMEM media supplemented with 10% FBS and 1% penicillin/streptomycin. Cas9 (1 μg) were complexed with crRNA (125 ng) and tracrRNA (125 ng) in one tube and incubated at room temperature for 5 minutes. Thereafter, ribonucleoprotein (RNP) was injected into the Hela/EGFP/RFP cells using electroporation (1350 V, 30 ms, 1 pulse). The cells were recovered with FBS-containing media for 48 hr, and the EGFP expression level in the cells was analyzed using flow cytometry (GUAVA, Millipore, USA).

### Example 2-4: Confirmation of Target Cleavage Activity (T7E1 Assay)

In order to confirm gene disruption level at the cellular level, a T7E1 assay was performed using the PCR-amplified target gene in the cells treated with RNP by the electroporation.

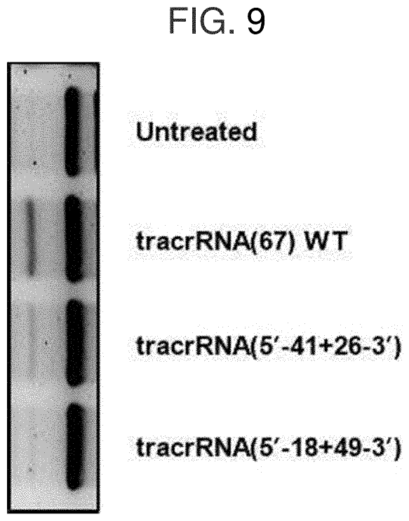

T7 endonuclease I (T7E1) is a structure-selective enzyme capable of detecting structural modifications in heteroduplex DNA, and the T7E1 assay was performed using a known method. Specifically, in order to detect gene editing by a CRISPR-Cas9, reagents were transfected into cells and genomic DNA around the target region was amplified by PCR for several days. A DNA extraction kit (MagListo 5M genomic DNA extraction kit, Bioneer, Korea) was used to prepare the genomic DNA of the RNP-injected cells, and the genomic DNA was PCR-amplified with Q5 Hot Start High-Fidelity 2× Master Mix (Engen mutation detection kit, New England Biolabs, USA) by following the manufacturer's protocol. The PCR products were digested at 37° C. for 15 minutes and analyzed on 2% agarose gel. PCR products are digested and recombined as the temperature rises and falls, and result in a DNA conformational change in which heteroduplex DNA is formed between amplicons having different lengths and may be recognized and cleaved by T7E1 when CRISPR-Cas9-induced non-homologous end-joining (NHEJ) occurs. Thereafter, the activity of CRISPR-Cas9 was compared by comparing the banding patterns of the cleavage products of the control and the experimental group in order to confirm the mutation frequency (FIG. 9 and the like).

### Example 2-5: Confirmation of Target Cleavage Activity (Sequencing and TIDE Analysis)

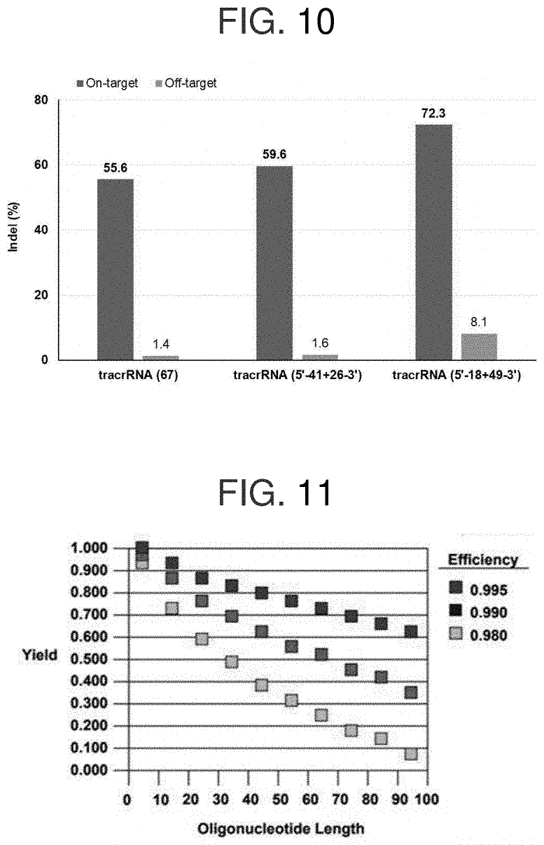

Genomic DNA was extracted from the cells, into which RNP was injected by the electroporation, using MagListo™ 5M Genomic DNA extraction kit (Bioneer. Korea), and then the on-target region and the off-target region were amplified by PCR. The sequences of the amplicons were analyzed by Sanger sequencing (Macrogen, Korea), and then the non-homologous end joining portion generated by CRISPR/Cas9 cleavage was confirmed through Tracking of Indels by DEcomposition (TIDE) analysis (FIG. 10 and the like).

### Example 3: Discovery of Excellent tracrRNA (67) Having Short Sequence and tracrRNA Including Fragment Thereof

It is widely known in the art that there are a large number of small-sized tracrRNAs of which the function is diminished among small-sized tracrRNAs obtained by cleaving a part of a wild-type tracrRNA. Accordingly, the present inventors have discovered an excellent tracrRNA (67 nt) having a short sequence from the wild-type tracrRNA and a fragment combination thereof in order to develop a novel CRISPR-Cas9 system exhibiting high sensitivity while utilizing a small tracrRNA or fragments thereof.

First, in order to confirm the in vitro gene cleavage activity of Example 2-1, a target region in the EGFP gene was set, and then a crRNA including a sequence capable of binding to the target DNA was obtained. In order to discover tracrRNAs including essential sequences having target DNA cleavage ability, various tracrRNAs having partially cleaved 3′ ends were obtained. Then, in order to identify the tracrRNA essential sequence, the tracrRNAs were divided into pieces based on the tracrRNA structure to prepare tracrRNAs including the respective stem-loops. RNP complexes were prepared using various tracrRNAs and then reacted with linear EGFP plasmids to measure the cleavage activity and derive the speed constant.

Among these, a tracrRNA fragment combination exhibiting activity similar to that of the existing wild-type tracrRNA was selected, and whether the combination was applicable to a 3-guide RNA system was confirmed. To this end, an additional target site in the EGFP gene was set, and in vitro Cas9 activity and speed constant were derived using the selected tracrRNA, and compared with those of the existing dual-guide RNA system. For comparison with the existing system, off-target activity analysis and target cleavage activity analysis were performed.

As a result, a tracrRNA (67) having the nucleotide sequence of SEQ ID NO. 1 was discovered as a tracrRNA having a short sequence and excellent practicality and economical efficiency while exhibiting a sensitivity and an effect similar to those of a CRISPR-Cas9 system including the existing wild-type tracrRNA.

In order to verify that a wild-type tracrRNA, fragments of the tracrRNA (67), and a combination of the fragments can also function as a tracrRNA, the target cleavage ability of CRISPR/Cas9 including several types of fragments of the tracrRNA (67) was confirmed.

As a result, when SEQ ID NO: 1 is divided into region A and region B, region A is divided into region 1 and region 2, and region B is divided into region 3 and region 4, it has been confirmed that B, B+1, B+2, or B+1+2 does not function as a tracrRNA when used as a tracrRNA, but A, A+3, A+4, or A+3+4 functions as a tracrRNA when used as a tracrRNA, and a CRISPR/Cas9 system including the A, A+3, A+4, or A+3+4 tracrRNA has target cleavage ability (FIG. 1).

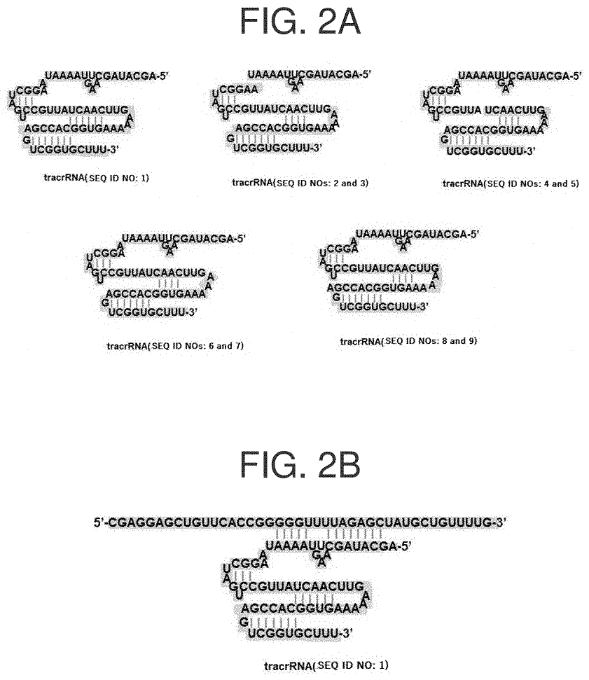

In addition, it has been confirmed that a dual-tracrRNA including two fragments of SEQ ID NO: 1 also has an excellent effect as a tracrRNA. The schematic diagrams of such tracrRNAs are illustrated in FIGS. 1 and 2.

From the results, it has been confirmed that a wild-type tracrRNA, fragments of tracrRNA (67), and a combination of the fragments can also function as a tracrRNA, and it has been confirmed that all of the tracrRNAs including the fragments essentially include a region consisting of the nucleotide sequence of SEQ ID NO: 2 as a region complementary to a crRNA. Consequently, it has been confirmed that the presence of region A (SEQ ID NO. 2) corresponding to the 18th nucleotide region from 5′ of SEQ ID NO: 1 is an essential element to operate as a tracrRNA.

### Example 4: Function Evaluation of tracrRNA Including tracrRNA (67) or Fragment Thereof

The present inventors evaluated the function of a CRISPR-Cas9 system having a tracrRNA system including the tracrRNA (67) or two fragments thereof using the method described in Example 2 above.

### Example 4-1. Evaluation on DNA Cleavage Ability (In Vitro Target Cleavage Assay)

The DNA cleavage ability of the tracrRNA (67) having the nucleotide sequence of SEQ ID NO: 1 and the tracrRNAs including two fragments thereof obtained in Example 3 was evaluated using the method of Example 2-1.

The function of each of four dual-tracrRNAs including a fragment consisting of the nucleotide sequence of SEQ ID NO: 2 and a fragment consisting of the nucleotide sequence of SEQ ID NO: 3 (tracrRNA (18+49)); a fragment consisting of the nucleotide sequence of SEQ ID NO: 4 and a fragment consisting of the nucleotide sequence of SEQ ID NO: 5 (tracrRNA (33+34)); a fragment consisting of the nucleotide sequence of SEQ ID NO: 6 and a fragment consisting of the nucleotide sequence of SEQ ID NO. 7 (tracrRNA (41+26)); and a fragment consisting of the nucleotide sequence of SEQ ID NO: 8 and a fragment consisting of the nucleotide sequence of SEQ ID NO: 9 (tracrRNA (49+18)) as two fragments of SEQ ID NO: 1 was evaluated.

As a result, it has been confirmed that the tracrRNA (67) having the nucleotide sequence of SEQ ID NO: 1 and the combination including two fragments thereof have an effect of cleaving the target region in vitro as the wild-type (WT). In particular, it has been confirmed that the tracrRNA (67), tracrRNA (49+18), tracrRNA (41+26), and tracrRNA (18+49) have the ability of cleaving the target region at a high speed (FIG. 3).

### Example 4-2. Evaluation on Extracellular Off-Target Side Effect

The off-target side effects of the tracrRNA (67), tracrRNA (41+26), and tracrRNA (18+49) among the five types of tracrRNAs obtained in Example 3 were evaluated using the method of Example 2-2. To this end, whether the function was the same as that of the wild-type tracrRNA was confirmed by comparing the degree of off-target effect of the tracrRNA (67), tracrRNA (41+26), and tracrRNA (18+49) with that of the wild-type tracrRNA.

As a result, it has been confirmed that the examples tracrRNA (67), tracrRNA (41+26), and tracrRNA (18+49) do not have a significant difference in off-target effect from the wild-type tracrRNA, and thus the short-length tracrRNA (67) or combinations of fragments thereof, tracrRNA (41+26), and tracrRNA (18+49) also do not have side effects of increasing the off-target effect (FIGS. 4 to 6).

### Example 4-3: Confirmation of Intracellular Target Cleavage Activity (Flow Cytometry, Western Blot, T7E1 Assay, and TIDE Analysis)

Representatively, the cleavage activity of the tracrRNA (67), tracrRNA (18+49), and tracrRNA (41+26) among the tracrRNAs obtained in Example 3 at the target site was evaluated using the methods of Examples 2-3 to 2-5.

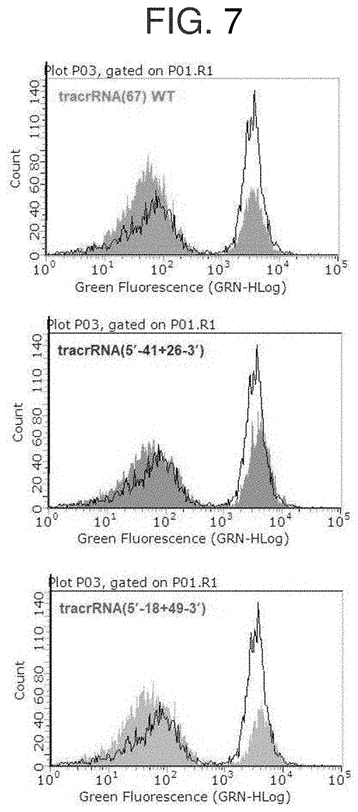

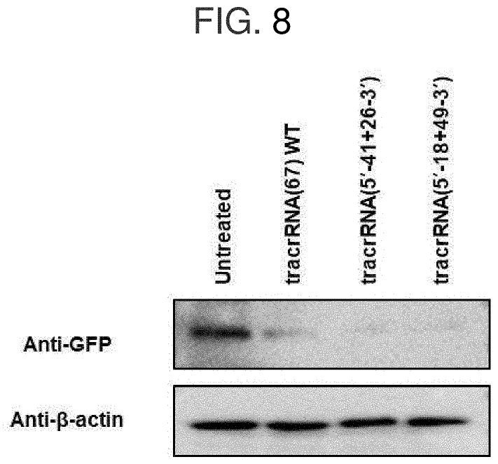

As a result, it has been confirmed that the tracrRNA (67), tracrRNA (41+26), and tracrRNA (18+49) all have the same target cleavage effect as the wild-type tracrRNA (FIG. 7: flow cytometry, FIG. 8: Western blot, FIG. 9: T7E1 assay, and FIG. 10: TIDE analysis).

From the above-described Examples, it has been confirmed that the tracrRNA (67) consisting of the nucleotide sequence of SEQ ID NO: 1, which is shorter than that of the wild-type tracrRNA (89 nucleotides), as well as a tracrRNA system including a fragment thereof maintain the same level of sensitivity as the wild-type tracrRNA.

Meanwhile, in fact, it is known that the synthesis yield decreases by about 40% when the length of the oligonucleotide is 50 nt or more, and the synthesis yield decreases to half or less when the length of the oligonucleotide is 80 nt or more (FIG. 11). In other words, it has been confirmed that a tracrRNA system including the tracrRNA (67) as well as a tracrRNA system including a fragment thereof are simpler than the existing system, can be mass-produced, and can thus be an effective system which can be practically applied and maintains the sensitivity.

### Example 5: tracrRNA Function Evaluation Method 2

In addition to the method of Example 2, the following tracrRNA function evaluation method and affinity evaluation method were used.

### Example 5-1: Confirmation of Target Cleavage Activity (Flow Cytometry)

In order to confirm the target cleavage activity at the cellular level, a Hela-EGFP cell line was utilized. HeLa/EGFP cells (1×10⁵) were seeded in 12-well plates and were cultured in DMEM media supplemented with 10% FBS and 1% penicillin/streptomycin. Cas9 (1 μg) was complexed with crRNA (125 ng) and tracrRNA (125 ng) in one tube and incubated at room temperature for 5 minutes. Then, ribonucleoprotein (RNP) was injected into the Hela/EGFP cells using electroporation (1350 V, 30 ms, 1 pulse). The cells were recovered with FBS-containing media for 48 hr and the EGFP expression level in the cells was analyzed using flow cytometry (GUAVA, Millipore, USA).

### Example 5-2: Confirmation of Target Cleavage Activity (Sequencing and TIDE Analysis)

The cells into which RNP was injected using electroporation for 48 hours were washed twice with phosphate-buffered saline (PBS), and the genomic DNA was extracted using MagListo™ 5M Genomic DNA extraction kit (Bioneer, Korea) according to the manufacturer's instruction. Thereafter, double-stranded DNA target/off-target region was amplified by PCR. The PCR was performed using Q5 High-Fidelity DNA polymerase (New England Biolabs Inc., Ipswich, Mass., USA) from the genomic DNA. The sequences of the amplicon were analyzed by Sanger sequencing (Macrogene, Korea), and then the non-homologous end joining portion generated by CRISPR/Cas9 cleavage was confirmed through Tracking of Indels by DEcomposition (TIDE) analysis.

### Example 6: Function Evaluation 2 of tracrRNA Including tracrRNA (67) or Fragment Thereof

### Example 6-1: Evaluation on DNA Cleavage Ability (In Vitro Cleavage Assay)

The DNA cleavage ability of tracrRNAs including two fragments of the tracrRNA (67) having the nucleotide sequence of SEQ ID NO: 1 obtained in Example 3 was evaluated and quantified using the method of Example 2-1.

The function of each of four dual-tracrRNAs including RNA consisting of the nucleotide sequence of SEQ ID NO: 2 and a fragment consisting of the nucleotide sequence of SEQ ID NO: 3 (tracrRNA (18+49)); a fragment consisting of the nucleotide sequence of SEQ ID NO: 4 and a fragment consisting of the nucleotide sequence of SEQ ID NO: 5 (tracrRNA (33+34)); a fragment consisting of the nucleotide sequence of SEQ ID NO: 6 and a fragment consisting of the nucleotide sequence of SEQ ID NO: 7 (tracrRNA (41+26)); and a fragment consisting of the nucleotide sequence of SEQ ID NO: 8 and a fragment consisting of the nucleotide sequence of SEQ ID NO: 9 (tracrRNA (49+18)) as two fragments of SEQ ID NO: 1 was evaluated.

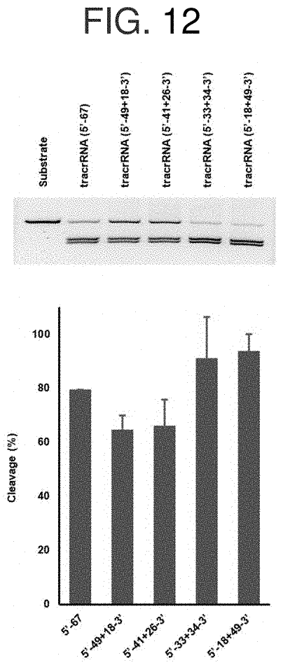

As a result, it has been confirmed that the cleavage ability (%) of the tracrRNA (49+18), tracrRNA (41+26), tracrRNA (33+34), and tracrRNA (18+49) was as excellent as that of the tracrRNA (67) (FIG. 12). It has been confirmed that the tracrRNA (67) has an effect similar to that of the wild-type (WT) in Examples described above, and thus the results suggest that the tracrRNA (49+18), tracrRNA (41+26), tracrRNA (33+34), and tracrRNA (18+49) also have an effect as excellent as that of the wild-type (WT).

### Example 6-2: Evaluation on DNA Cleavage Ability (In Vitro Off-Target Cleavage Assay)

The off-target cleavage of the four types of tracrRNA (49+18), tracrRNA (41+26), tracrRNA (33+34), and tracrRNA (18+49) was evaluated using the methods of Examples 2-1 and 2-2.

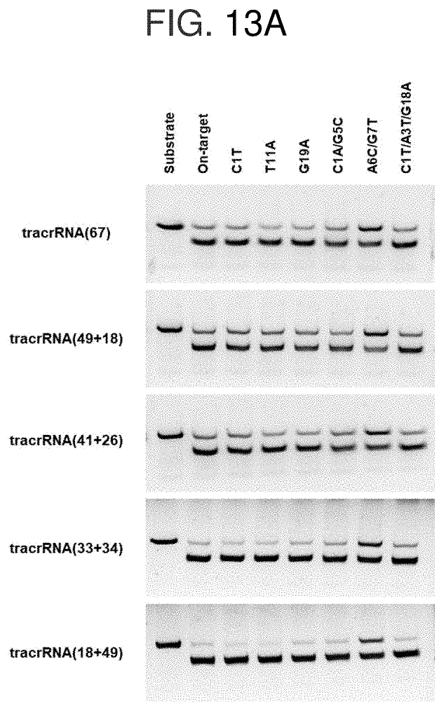

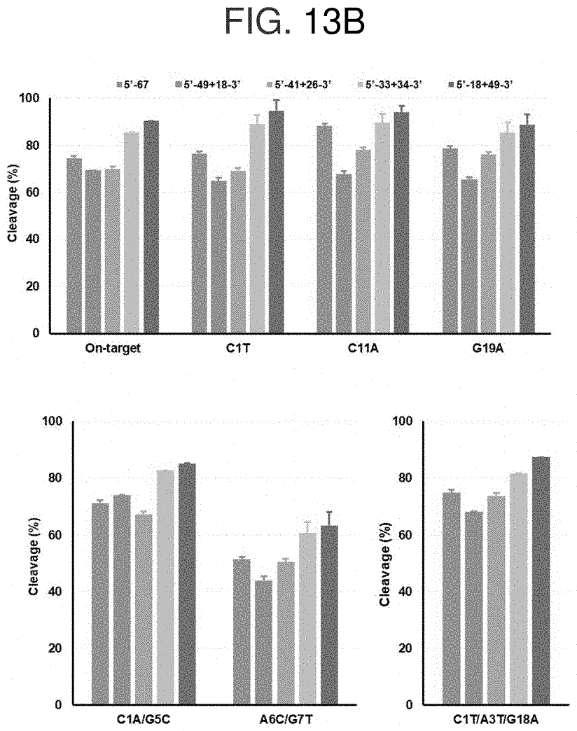

As a result, it has been confirmed that the four types of tracrRNA (49+18), tracrRNA (41+26), tracrRNA (33+34), and tracrRNA (18+49) all do not have off-target cleavage side effects, as with the tracrRNA (67) (FIGS. 13A and 13B).

### Example 6-3: Confirmation of Intracellular Target Cleavage Activity (Flow Cytometry, TIDE Assay)

The cleavage activity of the four types of tracrRNA (49+18), tracrRNA (41+26), tracrRNA (33+34), and tracrRNA (18+49) at the target site was evaluated using the methods of Example 5-1 and Example 5-2.

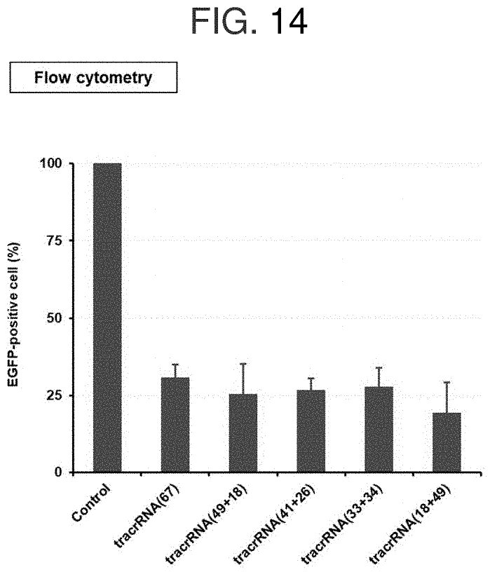

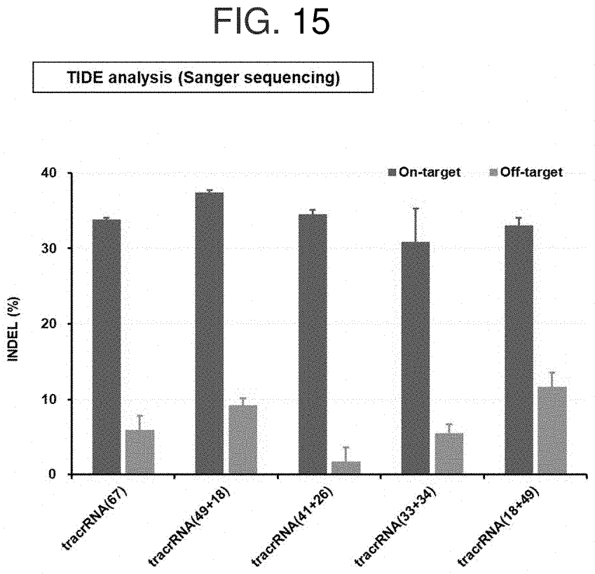

As a result, it has been confirmed that the four types of tracrRNA (49+18), tracrRNA (41+26), tracrRNA (33+34), and tracrRNA (18+49) all have a target cleavage effect as excellent as that of the tracrRNA (67) (FIG. 14: flow cytometry, and FIG. 15: TIDE analysis).

### Example 7: Discovery of tracrRNA Including Fragment of tracrRNA (67) and Having Nick in Stem-Loop 2

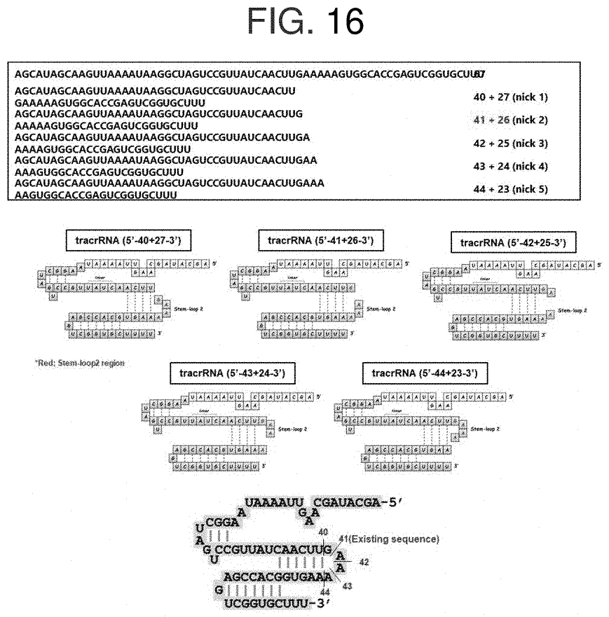

In addition to the fragments or combinations thereof discovered in Example 3, other fragments and combinations thereof were also discovered. In this process, the activity of different nick versions of the stem-loop 2 region was investigated, and the position of the nick and the structure of the prepared tracrRNA (40+27), tracrRNA (41+27), tracrRNA (42+25), tracrRNA (43+24), and tracrRNA (44+23) are illustrated in FIG. 16.

The tracrRNA (40+27) is a dual-tracrRNA of a fragment consisting of the nucleotide sequence of SEQ ID NO: 13 and a fragment consisting of the nucleotide sequence of SEQ ID NO: 14; the tracrRNA (41+26) is a dual-tracrRNA of a fragment consisting of the nucleotide sequence of SEQ ID NO: 6 and a fragment consisting of the nucleotide sequence of SEQ ID NO: 7; the tracrRNA (42+25) is a dual-tracrRNA of a fragment consisting of a nucleotide sequence of SEQ ID NO: 15 and a fragment consisting of a nucleotide sequence of SEQ ID NO: 16; the tracrRNA (43+24) is a dual-tracrRNA of a fragment consisting of the nucleotide sequence of SEQ ID NO: 17 and a fragment consisting of the nucleotide sequence of SEQ ID NO: 18; and the tracrRNA (44+23) is a dual-tracrRNA of a fragment consisting of the nucleotide sequence of SEQ ID NO: 19 and a fragment consisting of the nucleotide sequence of SEQ ID NO: 20. The SEQ ID NOs: 6, 7, and 13 to 20 are fragments of SEQ ID NO: 1, and SEQ ID NOs: 6, 13, 15, 17, and 19 among these include SEQ ID NO: 2.

### Example 8: Function Evaluation 3 of tracrRNA Including tracrRNA (67) or Fragment Thereof

### Example 8-1: Evaluation on DNA Cleavage Ability (In Vitro Cleavage Assay)

The DNA cleavage ability of the five types of tracrRNA (40+27), tracrRNA (41+27), tracrRNA (42+25), tracrRNA (43+24), and tracrRNA (44+23) obtained in Example 7 was evaluated and quantified using the method of Example 2-1.

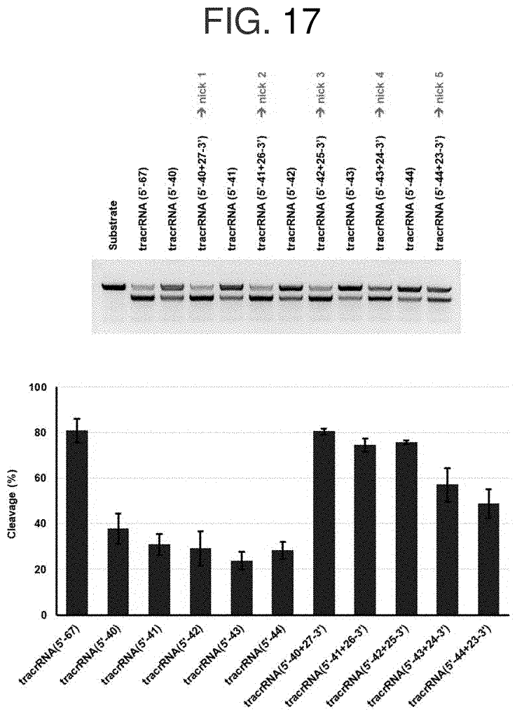

As a result, it has been confirmed that the five types of tracrRNA (40+27), tracrRNA (41+27), tracrRNA (42+25), tracrRNA (43+24), and tracrRNA (44+23) and the tracrRNA (5′-40), tracrRNA (5′-41), tracrRNA (5′-42), tracrRNA (5′-43), and tracrRNA (5′-44) corresponding to the 5′ fragments of the five tracrRNAs all have target cleavage ability (%). However, it has been confirmed that the cleavage ability of the five types of dual-tracrRNA including both the 5′ fragment and the 3′ fragment is superior to that of the tracrRNA (5′-40), tracrRNA (5′-41), tracrRNA (5′-42), tracrRNA (5′-43), and tracrRNA (5′-44), which are the 5′ fragments of the five tracrRNAs (FIG. 17).

### Example 8-2: Confirmation of Intracellular Target Cleavage Activity (Flow Cytometry, TIDE Assay)

The cleavage activity of the five types of tracrRNA (40+27), tracrRNA (41+27), tracrRNA (42+25), tracrRNA (43+24), and tracrRNA (44+23) at the target site was evaluated using the methods of Example 5-1 and Example 5-2.

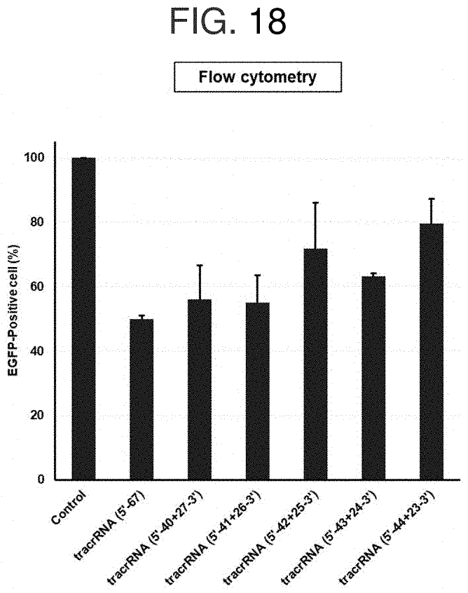

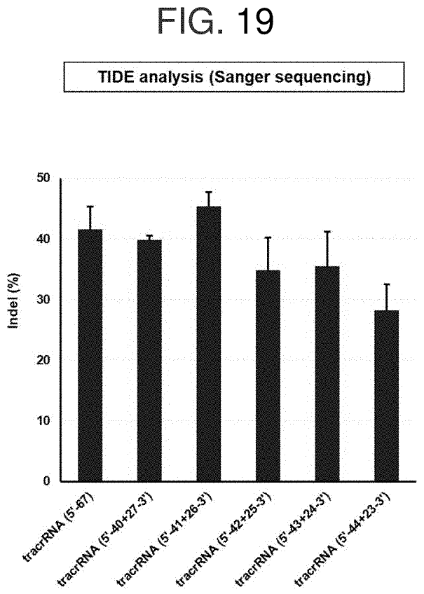

As a result, it has been confirmed that the tracrRNA (40+27), tracrRNA (41+27), tracrRNA (42+25), tracrRNA (43+24), and tracrRNA (44+23) all have an excellent target cleavage effect (FIG. 18: flow cytometry, and FIG. 19: TIDE analysis).

### Example 9: Discovery of tracrRNA Including Fragment of tracrRNA (67), in which Some or all Nucleotides of Stem-Loop 2 are Deleted

In addition to the fragment combinations discovered in Examples 3 and 7, other fragments and combinations thereof were discovered. In this process, the tracrRNA (5′-40), tracrRNA (5′-41), tracrRNA (5′-42), tracrRNA (5′-43), and tracrRNA (5′-44) corresponding to the 5′ fragments of the five tracrRNAs discovered in Example 7 and the tracrRNA (5′-23), tracrRNA (5′-24), tracrRNA (5′-25), tracrRNA (5′-26), and tracrRNA (5′-27) corresponding to the 3′ fragments of the five tracrRNAs were used in different combinations to prepare dual-tracrRNAs in which some nucleotides of the stem-loop 2 region were deleted or fragments thereof.

### Example 10: Function Evaluation 4 of tracrRNA Including tracrRNA (67) or Fragment Thereof—Evaluation on DNA Cleavage Ability (In Vitro Cleavage Assay)

The DNA cleavage ability of the tracrRNA(5′-40), tracrRNA(40+27), tracrRNA(40+26), tracrRNA(40+25), tracrRNA(40+24), tracrRNA(40+23), tracrRNA(5′-41), tracrRNA(41+26), tracrRNA(41+25), tracrRNA(41+24), tracrRNA(41+23), tracrRNA(5′-42), tracrRNA(42+25), tracrRNA(42+24), tracrRNA(42+23), tracrRNA(5′-43), tracrRNA(43+24), tracrRNA(43+23), tracrRNA (5′-44), and tracrRNA (44+23) obtained in Example 9 was evaluated and quantified using the method of Example 2-1.

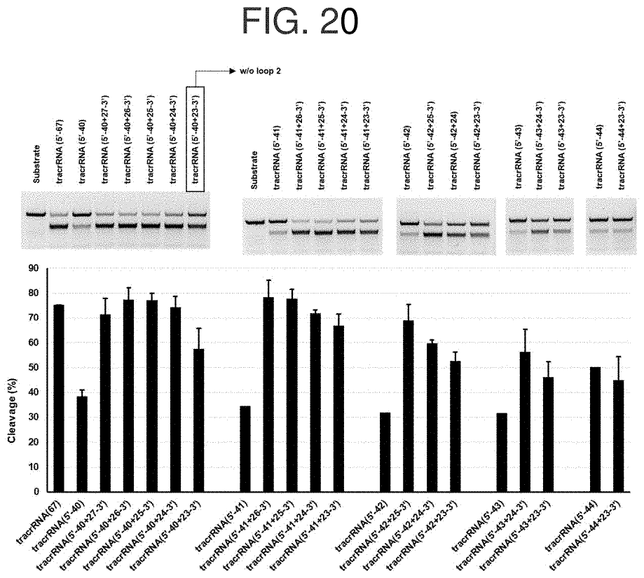

As a result, it has been confirmed that the evaluated tracrRNAs all have target cleavage ability (%). However, it has been confirmed that the cleavage ability of the five types of dual-tracrRNAs including both the 5′ fragment and the 3′ fragment is superior to that of the 5′ fragments of the tracrRNAs and the dual-tracrRNAs in which stem-loop 2 was maintained have superior cleavage ability compared to the dual-tracrRNAs in which a part or the whole of stem-loop 2 was deleted (FIG. 20).

### Example 11: Discovery of tracrRNA Including Fragment of tracrRNA (67), in which Stem-Loop 2 is Deleted and Adjacent Nucleotides of Stem-Loop 2 are Substituted

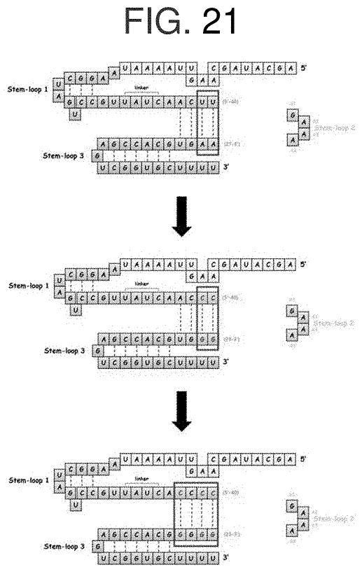

In addition to the fragment combinations discovered in Examples 3, 7, and 9, other fragment combinations in which stem-loop 2 is deleted and some of its adjacent nucleotides are substituted were discovered. In this process, two or four 3′-terminal nucleotides of the 5′ fragment of the stem-loop 2 deleted tracrRNA (40+23) were substituted with cytosine (C); two or four 5′-terminal nucleotides of the 3′ fragment of the tracrRNA (40+23) were substituted with guanine (G); four 3′-terminal nucleotides of the 5′ fragment of the tracrRNA (40+23) were substituted with F-C (2′-fluoro-cytosine); and/or four 5′-terminal nucleotides of the 3′ fragment of the tracrRNA (40+23) were substituted with F-G (2′-fluoro-guanine). A schematic diagram of its structure is illustrated in FIG. 21.

### Example 12: Function Evaluation 5 of tracrRNA Including tracrRNA (67) or Fragment Thereof

### Example 12-1: Evaluation on DNA Cleavage Ability (In Vitro Cleavage Assay)

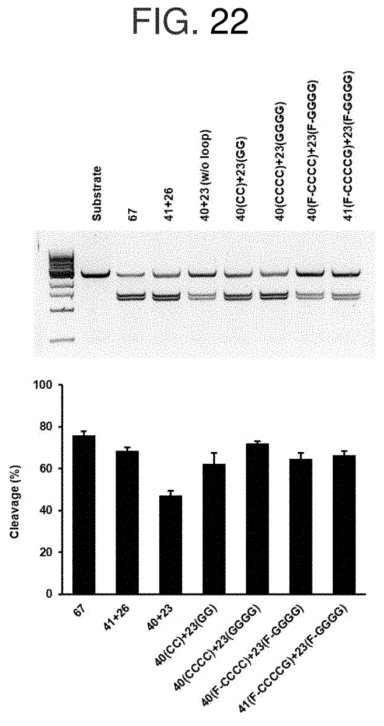

The DNA cleavage ability of the tracrRNAs obtained in Example 11 was evaluated and quantified using the method of Example 2-1. As a result, it has been confirmed that the cleavage ability increases as the substitution with C, F-C, G, and/or F-G increases (FIG. 22).

### Example 12-2: Confirmation of Intracellular Target Cleavage Activity (Flow Cytometry, TIDE Assay)

The cleavage activity of the tracrRNAs obtained in Example 11 at the target site was evaluated using the methods of Examples 5-1 and 5-2.

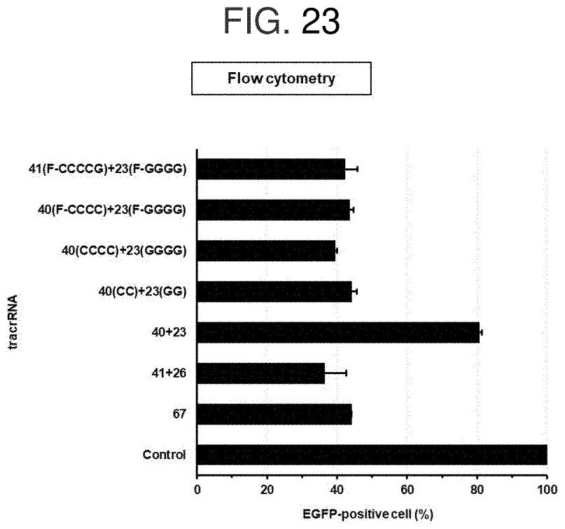

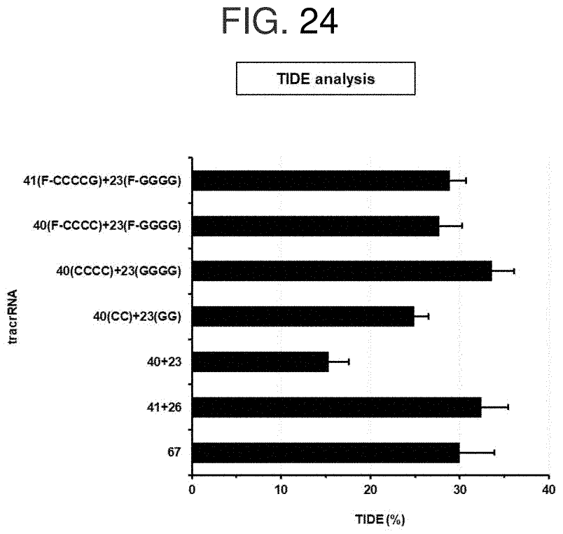

As a result, it has been confirmed that the tracrRNA (40(CC)+23(GG)), tracrRNA (40(CCCC)+23(GGGG)), tracrRNA (40(F-CCCC)+23(F-GGGG)), and tracrRNA (41(CCCCG)+23(FGGGG)), which are dual-tracrRNAs in which four to eight nucleotides adjacent to the stem-loop 2 position are substituted in the dual-tracrRNA (40+23) from which stem-loop 2 has been removed, have superior target cleavage activity, and the target cleavage activity is as excellent as the target cleavage activity of the tracrRNA (67) and dual-tracrRNA (41+26) from which stem-loop 2 has not been removed (FIG. 23: flow cytometry, and FIG. 24: TIDE analysis).

From the results, it has been confirmed that various types of tracrRNA including a tracrRNA containing the nucleotide sequence of SEQ ID NO: 2 have excellent target cleavage activity in a CRISPR-Cas9 system, and can thus be utilized in a highly useful gene editing system.

Based on the above description, it will be understood by those skilled in the art that the present invention may be implemented in a different specific form without changing the technical spirit or essential characteristics thereof. Therefore, it should be understood that the above embodiment is not limitative, but illustrative in all aspects. The scope of the present invention is defined by the appended claims rather than by the description preceding them, and thus all changes and modifications that fall within metes and bounds of the claims or equivalents of such metes and bounds are therefore intended to be embraced by the claims.

1. A trans-activating CRISPR RNA (tracrRNA) comprising a nucleotide sequence of SEQ ID NO: 2.

2. The trans-activating CRISPR RNA (tracrRNA) according to claim 1, wherein the nucleotide sequence of SEQ ID NO: 2 is a fragment of a nucleotide sequence of SEQ ID NO: 1 and is a region binding to a CRISPR RNA (crRNA).

3. The trans-activating CRISPR RNA (tracrRNA) according to claim 1, wherein the tracrRNA comprises any one nucleotide sequence selected from the group consisting of SEQ ID NOs: 4, 6, 8, 13, 15, 17, 19, 21, 23, 25, and 27.

4. The trans-activating CRISPR RNA (tracrRNA) according to claim 1, wherein the tracrRNA further comprises one or two fragments of a nucleotide sequence of SEQ ID NO: 1.

5. The trans-activating CRISPR RNA (tracrRNA) according to claim 4, wherein the fragment of the nucleotide sequence of SEQ ID NO: 1 consists of any one nucleotide sequence selected from the group consisting of SEQ ID NOs: 3, 5, 7, 9 to 11, 14, 16, 18, 20, 22, 24, and 26.

6. The trans-activating CRISPR RNA (tracrRNA) according to claim 4, wherein the tracrRNA comprises a fragment consisting of a nucleotide sequence of SEQ ID NO: 2 and a fragment consisting of a nucleotide sequence of SEQ ID NO: 3;
a fragment consisting of a nucleotide sequence of SEQ ID NO: 4 and a fragment consisting of a nucleotide sequence of SEQ ID NO: 5;
a fragment consisting of a nucleotide sequence of SEQ ID NO: 6 and a fragment consisting of a nucleotide sequence of SEQ ID NO: 7;
a fragment consisting of the nucleotide sequence of SEQ ID NO: 8 and a fragment consisting of the nucleotide sequence of SEQ ID NO: 9;
a fragment consisting of the nucleotide sequence of SEQ ID NO: 13 and a fragment consisting of the nucleotide sequence of SEQ ID NO: 14;
a fragment consisting of the nucleotide sequence of SEQ ID NO: 15 and a fragment consisting of the nucleotide sequence of SEQ ID NO: 16;
a fragment consisting of the nucleotide sequence of SEQ ID NO: 17 and a fragment consisting of the nucleotide sequence of SEQ ID NO: 18;
a fragment consisting of the nucleotide sequence of SEQ ID NO: 19 and a fragment consisting of the nucleotide sequence of SEQ ID NO: 20;
a fragment consisting of the nucleotide sequence of SEQ ID NO: 13 and a fragment consisting of the nucleotide sequence of SEQ ID NO: 7;
a fragment consisting of the nucleotide sequence of SEQ ID NO: 13 and a fragment consisting of the nucleotide sequence of SEQ ID NO: 16;
a fragment consisting of the nucleotide sequence of SEQ ID NO: 13 and a fragment consisting of the nucleotide sequence of SEQ ID NO: 18;
a fragment consisting of the nucleotide sequence of SEQ ID NO: 13 and a fragment consisting of the nucleotide sequence of SEQ ID NO: 20;
a fragment consisting of the nucleotide sequence of SEQ ID NO: 6 and a fragment consisting of the nucleotide sequence of SEQ ID NO: 16;
a fragment consisting of the nucleotide sequence of SEQ ID NO: 6 and a fragment consisting of the nucleotide sequence of SEQ ID NO: 18;
a fragment consisting of the nucleotide sequence of SEQ ID NO: 6 and a fragment consisting of the nucleotide sequence of SEQ ID NO: 20;
a fragment consisting of the nucleotide sequence of SEQ ID NO: 15 and a fragment consisting of the nucleotide sequence of SEQ ID NO: 18;
a fragment consisting of the nucleotide sequence of SEQ ID NO: 15 and a fragment consisting of the nucleotide sequence of SEQ ID NO: 20;
a fragment consisting of the nucleotide sequence of SEQ ID NO: 17 and a fragment consisting of the nucleotide sequence of SEQ ID NO: 20;
a fragment consisting of the nucleotide sequence of SEQ ID NO: 21 and a fragment consisting of the nucleotide sequence of SEQ ID NO: 22;
a fragment consisting of the nucleotide sequence of SEQ ID NO: 23 and a fragment consisting of the nucleotide sequence of SEQ ID NO: 24;
a fragment consisting of the nucleotide sequence of SEQ ID NO: 25 and a fragment consisting of the nucleotide sequence of SEQ ID NO: 26; and/or
a fragment consisting of the nucleotide sequence of SEQ ID NO: 27 and a fragment consisting of the nucleotide sequence of SEQ ID NO: 26;

7. A guide RNA (gRNA) comprising the tracrRNA according to claim 1.

8. The gRNA according to claim 7, wherein the gRNA comprises one or more modified nucleotides.

9. The gRNA according to claim 8, wherein the modified nucleotide is any one selected from the group consisting of 2′-O-methyl RNA (2′-OMe RNA), 2′-O-methoxyethyl RNA (2′-MOE RNA), 2′-fluoro RNA (2′-F RNA), phosphorothioate RNA (PS RNA), 2′-amino RNA (2′-NH₂ RNA), 2′-fluoro arabinose nucleic acid (FANA), 4′-thio RNA (4′-S RNA), locked nucleic acid (LNA), threose nucleic acid (TNA), phosphorothioate DNA (PS DNA), DNA, peptide nucleic acid (PNA), phosphorodiamidate morpholino oligonucleotide (PMO), hexitol nucleic acid (HNA), cyclohexene nucleic acid (CeNA), and arabinose nucleic acid (ANA).

10. The gRNA according to claim 7, wherein the gRNA further comprises a CRISPR RNA (crRNA).

11. The gRNA according to claim 10, wherein the crRNA consists of a nucleotide sequence of SEQ ID NO: 12.

12. A CRISPR-Cas9 system comprising the tracrRNA according to claim 1.

13. A CRISPR-Cas9 system comprising the tracrRNA according to claim 3.

14. A CRISPR-Cas9 system comprising the tracrRNA according to claim 5.

15. A kit for DNA expression inhibition and DNA targeting, the kit comprising the tracrRNA according to claim 1 or a DNA encoding the tracrRNA; a crRNA or a DNA encoding the crRNA; and a Cas9 polypeptide or a polynucleotide encoding the Cas9 polypeptide.

16. A kit for DNA expression inhibition and DNA targeting, the kit comprising the tracrRNA according to claim 3 or a DNA encoding the tracrRNA; a crRNA or a DNA encoding the crRNA; and a Cas9 polypeptide or a polynucleotide encoding the Cas9 polypeptide.

17. A kit for DNA expression inhibition and DNA targeting, the kit comprising the tracrRNA according to claim 5 or a DNA encoding the tracrRNA; a crRNA or a DNA encoding the crRNA; and a Cas9 polypeptide or a polynucleotide encoding the Cas9 polypeptide.

18. A kit for gene editing, the kit comprising the tracrRNA according to claim 1 or a DNA encoding the tracrRNA; a crRNA or a DNA encoding the crRNA; and a Cas9 polypeptide or a polynucleotide encoding the Cas9 polypeptide.

19. A kit for gene editing, the kit comprising the tracrRNA according to claim 3 or a DNA encoding the tracrRNA; a crRNA or a DNA encoding the crRNA; and a Cas9 polypeptide or a polynucleotide encoding the Cas9 polypeptide.

20. A kit for gene editing, the kit comprising the tracrRNA according to claim 3 or a DNA encoding the tracrRNA; a crRNA or a DNA encoding the crRNA; and a Cas9 polypeptide or a polynucleotide encoding the Cas9 polypeptide.


SEQUENCE LISTING (27 sequences)

SEQ ID NO: 1 - RNA, 67 nt, Artificial sequence
  tracrRNA
agcauagcaaguuaaaauaaggcuaguccguuaucaacuugaaaaaguggcaccgagucggugcuuu

SEQ ID NO: 2 - RNA, 18 nt, Artificial Sequence
  tracrRNA
agcauagcaaguuaaaau

SEQ ID NO: 3 - RNA, 49 nt, Artificial Sequence
  tracrRNA
aaggcuaguccguuaucaacuugaaaaaguggcaccgagucggugcuuu

SEQ ID NO: 4 - RNA, 33 nt, Artificial Sequence
  tracrRNA
agcauagcaaguuaaaauaaggcuaguccguua

SEQ ID NO: 5 - RNA, 34 nt, Artificial Sequence
  tracrRNA
ucaacuugaaaaaguggcaccgagucggugcuuu

SEQ ID NO: 6 - RNA, 41 nt, Artificial Sequence
  tracrRNA
agcauagcaaguuaaaauaaggcuaguccguuaucaacuug

SEQ ID NO: 7 - RNA, 26 nt, Artificial Sequence
  tracrRNA
aaaaaguggcaccgagucggugcuuu

SEQ ID NO: 8 - RNA, 49 nt, Artificial Sequence
  tracrRNA
agcauagcaaguuaaaauaaggcuaguccguuaucaacuugaaaaagug

SEQ ID NO: 9 - RNA, 18 nt, Artificial Sequence
  tracrRNA
gcaccgagucggugcuuu

SEQ ID NO: 10 - RNA, 15 nt, Artificial Sequence
  tracrRNA
aaggcuaguccguua

SEQ ID NO: 11 - RNA, 16 nt, Artificial Sequence
  tracrRNA
ucaacuugaaaaagug

SEQ ID NO: 12 - RNA, 42 nt, Artificial Sequence
  crRNA
cgaggagcuguucaccggggguuuuagagcuaugcuguuuug

SEQ ID NO: 13 - RNA, 40 nt, Artificial Sequence
  tracrRNA
agcauagcaaguuaaaauaaggcuaguccguuaucaacuu

SEQ ID NO: 14 - RNA, 27 nt, Artificial Sequence
  tracrRNA
gaaaaaguggcaccgagucggugcuuu

SEQ ID NO: 15 - RNA, 42 nt, Artificial Sequence
  tracrRNA
agcauagcaaguuaaaauaaggcuaguccguuaucaacuuga

SEQ ID NO: 16 - RNA, 25 nt, Artificial Sequence
  tracrRNA
aaaaguggcaccgagucggugcuuu

SEQ ID NO: 17 - RNA, 43 nt, Artificial Sequence
  tracrRNA
agcauagcaaguuaaaauaaggcuaguccguuaucaacuugaa

SEQ ID NO: 18 - RNA, 24 nt, Artificial Sequence
  tracrRNA
aaaguggcaccgagucggugcuuu

SEQ ID NO: 19 - RNA, 44 nt, Artificial Sequence
  tracrRNA
agcauagcaaguuaaaauaaggcuaguccguuaucaacuugaaa

SEQ ID NO: 20 - RNA, 23 nt, Artificial Sequence
  tracrRNA
aaguggcaccgagucggugcuuu

SEQ ID NO: 21 - RNA, 40 nt, Artificial Sequence
  tracrRNA
agcauagcaaguuaaaauaaggcuaguccguuaucaaccc

SEQ ID NO: 22 - RNA, 23 nt, Artificial Sequence
  tracrRNA
ggguggcaccgagucggugcuuu

SEQ ID NO: 23 - RNA, 40 nt, Artificial Sequence
  tracrRNA
agcauagcaaguuaaaauaaggcuaguccguuaucacccc

SEQ ID NO: 24 - RNA, 23 nt, Artificial Sequence
  tracrRNA
ggggggcaccgagucggugcuuu

SEQ ID NO: 25 - RNA, 40 nt, Artificial Sequence; identical to SEQ ID NO: 23
  tracrRNA
  misc_feature (37)..(37): C is 2′-fluoro-cytosine

SEQ ID NO: 26 - RNA, 23 nt, Artificial Sequence
  tracrRNA
  misc_feature (1)..(1): G is 2′-fluoro-guanine
ggggggcaccgagucggugcuuu

SEQ ID NO: 27 - RNA, 41 nt, Artificial Sequence
  tracrRNA
  misc_feature (37)..(37): C is 2′-fluoro-cytosine
agcauagcaaguuaaaauaaggcuaguccguuaucaccccg